(19) United States
(12) Patent Application Publication
(10) Pub. No.: US 2003/0215839 A1
(43) Pub. Date: Nov. 20, 2003

(54) Methods and means for identification of gene features

(76) Inventors: Peter Lonnerberg, Stockholm (SE); Mats Oldin, Stockholm (SE); Sten Linnarsson, Stockholm (SE); Patrik Ernfors, Stockholm (SE)

Correspondence Address: NIXON & VANDERHYE, PC, 1100 N GLEBE ROAD, 8TH FLOOR, ARLINGTON, VA, 22201-4714, US

(21) Appl. No.: 10/352,255

(22) Filed: Jan. 28, 2003

Related U.S. Application Data
(60) Provisional application No. 60/352,245, filed on Jan. 29, 2002.

(51) Int. Cl. C12Q 1/68; G06F 19/00; G01N 33/48; G01N 33/50

(52) U.S. Cl. 435/6; 702/20

(57) ABSTRACT

Identification of gene variants, and in particular identification of differences between sequence variants that occur in a population of nucleic acid molecules, especially identification or discovery of polyA site usage, or determination of polyA site usage in a nucleic acid sample, and gene variants arising from alternative polyA sites.

The present invention relates to identification of gene variants. In particular the invention provides for identification of differences between sequence variants that occur in a population of nucleic acid molecules. In particular embodiments, the present invention relates to identification or discovery of polyA site usage, or determination of polyA site usage in a nucleic acid sample, and gene variants arising from alternative polyA sites.

### BRIEF DESCRIPTION OF THE FIGURES

FIG. 1 illustrates an embodiment of the present invention involving discovery of polyadenylation sites. Given a gene with two candidate poly(A) sites, and given three gene profiles produced in this case by restriction enzyme cleavage with three different enzymes, the appearance of peaks corresponding to the candidate poly(A) sites provides direct experimental evidence for their existence.

FIG. 2 outlines an approach to production of signals for transcribed mRNA in a sample, employing a Type II restriction enzyme (HaeII).

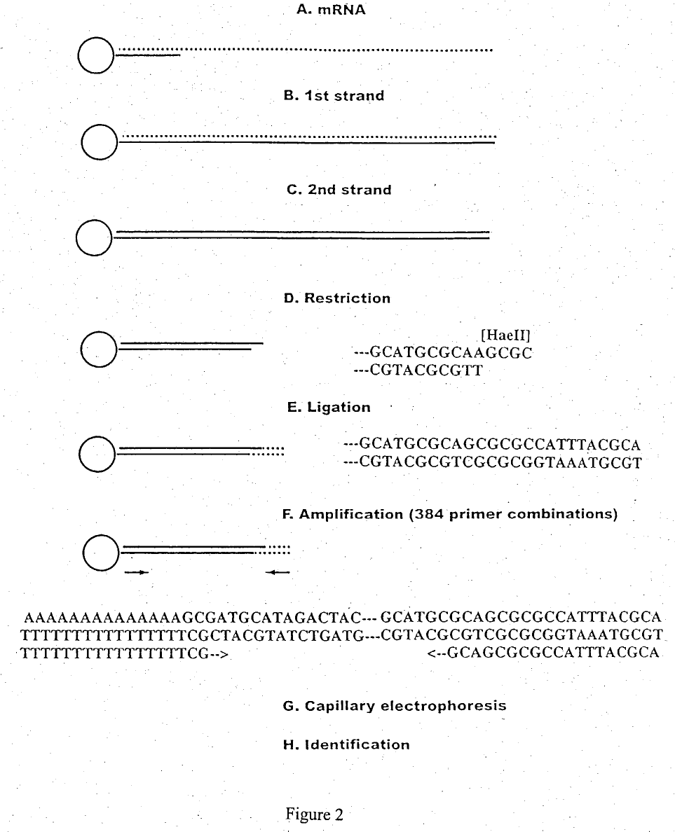

FIG. 3 outlines an approach to production of signals for transcribed mRNA in a sample, employing a Type IIS restriction enzyme (FokI).

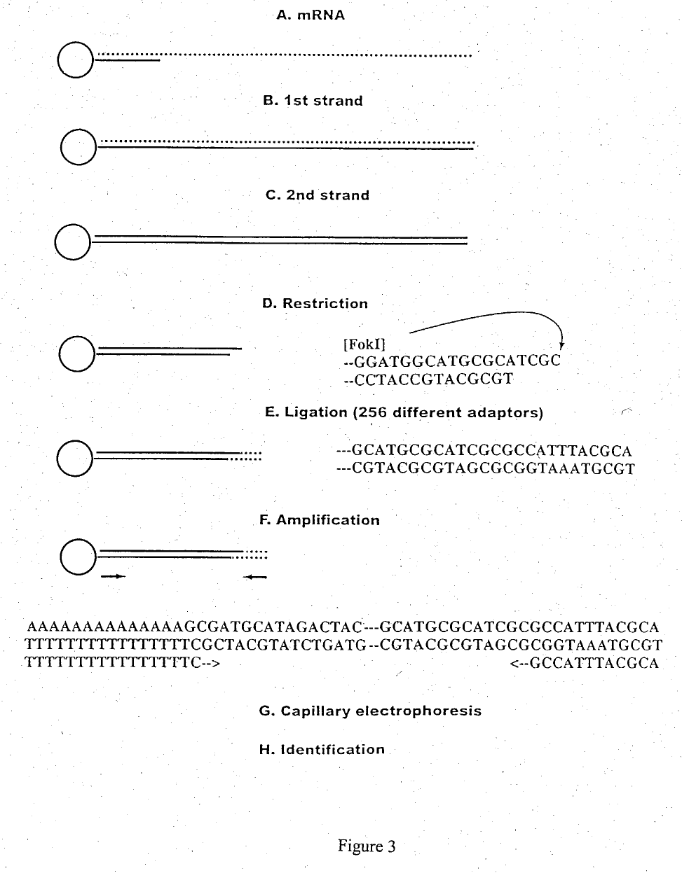

FIG. 4 shows the results of an experiment assessing specificity of ligation for an adaptor blocked on one strand. A single template oligonucleotide was used, having a four base pair single-stranded overhang, and adaptors were designed having a single stranded region exactly complementary to this, or with 1, 2 or 3 mismatches. Adaptors were ligated to the template oligonucleotide, and the products were amplified using PCR.

FIG. 5 outlines generation of signals for gene fragments corresponding to transcribed mRNA molecules present in a sample. Steps I to VII are shown:

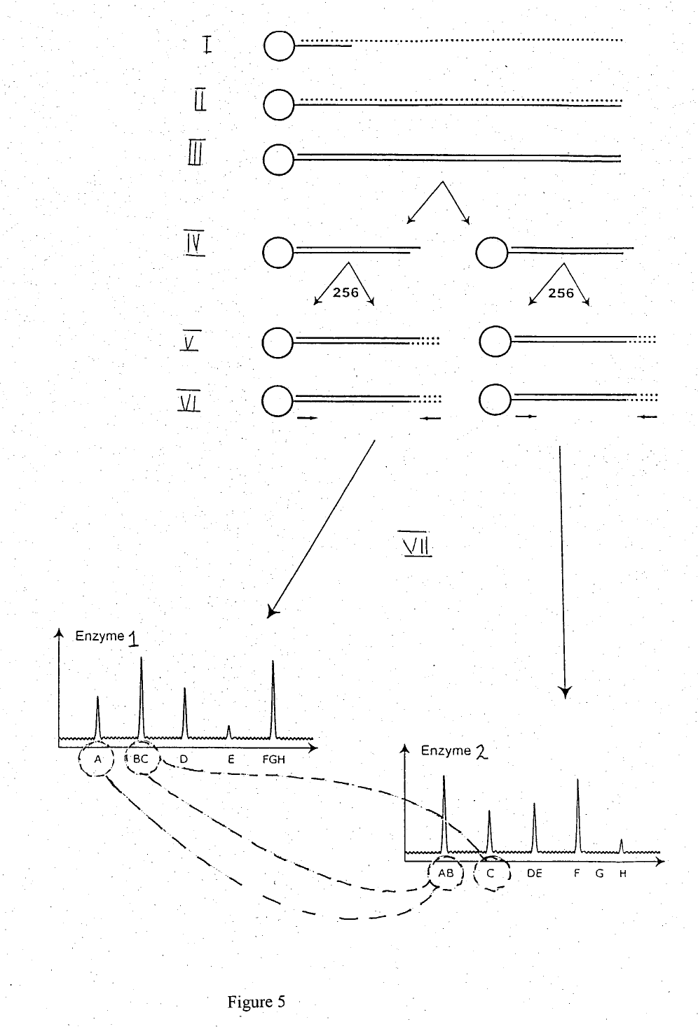

In step I, mRNA is captured on magnetic beads carrying an oligo-dT tail.

In step II, a complementary DNA strand is synthesized, still attached to the beads.

In step III, the mRNA is removed, and a second cDNA strand is synthesized. The double-stranded cDNA remains covalently attached to the beads.

In step IV, the double-stranded cDNA is split into two separate pools. Each pool is digested with a different restriction enzyme. The sequence of cDNA corresponding to the 3′ end of the mRNA remains attached to the beads.

In step V, adaptors are ligated to the digested end of the cDNA. In this embodiment of the invention, 256 different adaptors are ligated in 256 separate reactions. Also in this embodiment of the invention, the adaptors are blocked on one strand, so that PCR proceeds only from the other strand.

In step VI, each of the fractions is amplified with a single PCR primer pair.

In step VII, the PCR products are subject to capillary electrophoresis. This produces a independent pattern or set of signals for each of the pools, i.e. first and second populations of gene fragments provided by digestion of cDNA's by each of first and second different restriction enzymes.

In a few years the sequences of the human and rodent genomes will be complete. A more complex task is the identification and characterization of the transcriptome, the full set of genes expressed as messenger RNAs (mRNAs) from the genome, and that ultimately through translation into proteins control the development and proper function of the cells in an organism.

An important aspect of understanding gene action in the cell is to understand the regulation of transcription of the mRNAs from the genes. This is controlled by a complex set of enhancers and silencers binding to regulatory DNA sequences located mainly in the non-coding regions upstream and downstream of the protein-encoding portion of mRNAs. Many of these regulatory sequences are not precisely defined, which makes their detection difficult.

In later years it has been realized that the translation of mRNAs to protein is also regulated by a set of regulatory proteins binding to the 5′ and 3′ region of mRNAs. (reviewed by Macdonald et al. 2001). A further feature of mRNAs that has proved important for translational regulation is the use of alternative poyadenylation sites (pAsites) when defining the 3′ end of mRNAs. (For a few examples, see Touriol et al., 1999; Goldmann et al., 1999). As much as 22% of murine and 44% of human investigated genes show from two to nine alternative pAsites (Pauws et al., 2001).

The choice of pAsite determines which regulatory sequence elements are included in the downstream part of the mRNA, and also affects mRNA half-life. The available data on pAsite usage is poor due to the limitations of current pAsite determination methods, and hence it is difficult to make general conclusion on this translation regulation. For this reason, it is desirable to find better ways to determine the repertoire of pAsites of the transcriptome in various cell types and conditions.

So far, two methods have been used to investigate the 3′ ends of mRNAs:

1. Direct sequencing of cloned mRNAs.

By specifically cloning and sequencing 3′ ends of mRNAs from cell samples knowledge of pAsites can be accumulated. Major limitations of this method is that it is very labour intensive, and that artefact are quite common, so that the same 3′ end has to be found several times to be considered true. Furthermore, uncommon pAsites will be represented correspondingly seldomly among the cloned sequences, resulting in huge cloning projects to obtain results for but a few selected genes.

2. Computerized sequence searches for pAsite specific sequences.

Several efforts has been made to use the available knowledge of pAsite consensus sequences with or without EST clustering algorithms in computer algorithms to automatically finds likely pAsites in genomic or EST sequences (Tabaska and Zhang, 1999; Kan et al., 2001). Unfortunately, sequences specifying pAsites are surprisingly diverse (Beaudoing et al., 2000), especially for genes with alternative pAsites, and no reliable consensus sequence has been defined. Thus, the predictions from current computer algorithms are far from conclusive, and need to be confirmed by mRNA sequencing, again resulting in huge sequencing projects for whole-transcriptome analysis.

The present invention uses combinatorial identification to address these shortcomings. Length and/or partial sequence information obtained for a set of fragments—where each gene is represented by more than one fragment—is used to identify in a database those genes (or other sequences) which produced the observed fragments. The key to combinatorial identification is that each gene is seen more than once. This has the consequence that, even though one may find multiple candidate genes for each fragment (as in SAGE), there is collectively enough information to unambiguously identify each gene's contribution to a particular fragment.

One example of combinatorial identification is described in patent applications GB0018016.6 and PCT/IB01/01539, and further herein.

Generally, in performing embodiments of this method, double-stranded cDNA is generated from mRNA in a sample. This double-stranded cDNA is subject to restriction enzyme digestion to provide digested double-stranded cDNA molecules, each having a cohesive end provided by the restriction enzyme digestion.

In the present invention, information is gathered for the length of gene fragments based on how far the site of restriction enzyme digestion is from polyA and on partial sequence information. The combination of length and partial sequence information for each gene fragment provides a signal for that gene fragment, and a dataset of signals for populations of gene fragments may be generated. As discussed, length of nucleic acid molecules may be determined using standard electrophoretic techniques. Partial sequence information may be obtained by knowledge of the recognition site for the restriction enzyme, and also by means of differential amplification of digested fragments employing different adapters that anneal to gene fragments with an end resulting from the restriction enzyme digest depending on the base or bases at that end.

Thus, for example, a population of adaptor oligonucleotides (adaptors) may be ligated to the digested end of each of the digested double-stranded cDNA molecules, thereby providing double-stranded template cDNA molecules each comprising a first strand and a second strand, wherein the first strand of the double-stranded template cDNA molecules each comprise a 3′ terminal adaptor oligonucleotide and the second strand of the double-stranded template cDNA molecules each comprise a 3′ terminal polyA sequence.

These double-stranded template cDNA molecules may be purified, to provide a population of cDNA fragments having a sequence complementary to a 3′ end of an mRNA.

Purification of the double-stranded template cDNA molecules may be achieved by any suitable means available to the skilled person. For example, the polyA or polyT sequence at one end of the cDNA molecule may be tagged with biotin, allowing purification of these double-stranded template cDNA molecules by binding to streptavadin-coated beads. Alternatively, isolation of these double-stranded template cDNA molecules may be achieved by hybridisation selection, dependent on binding to an oligoT and/or oligoA probe, prior to PCR.

Preferably, digested double-stranded cDNA molecules comprising a strand having a 3′ terminal polyA sequence are purified prior to ligating the adaptor oligonucleotides. This has the advantage of preventing non-specific ligation of adaptors. Again, this may employ any of the methods available to the skilled person, including purification by biotin tagging, as described above.

In preferred embodiments, the 3′ ends of the cDNA sequence are immobilised prior to restriction digestion. Thus, one end of the cDNA generated from the mRNA is anchored to a solid support (such as beads, e.g. magnetic or plastic, or any other solid support that can be retained while washing, for instance by centrifugation or magnetism, or a microfabricated reaction chamber with sub-chambers for the subdivision procedure, where chemicals are washed through the chambers) by means of oligoT at the 5′ end—complementary to polyA originally at the 3′ end of the mRNA molecules. The other end of the cDNA sequence is subject to restriction enzyme digestion, and an adaptor is ligated to the free (digested) end. Purification of the above described digested double-stranded cDNA molecules or double-stranded template cDNA molecules may thus be achieved by washing away excess materials, while retaining the desired molecules on the solid support.

PCR may be performed using primers that anneal at the ends of the cDNA—one designed to anneal to the adaptor at the 3′ end of one strand of the cDNA, the other containing oligodT to anneal to polyA at the 3′ end of the other strand of the cDNA (corresponding to the original polyA in the mRNA). For use with a Type II enzyme, each primer includes a variable nucleotide or sequence of nucleotides that will amplify a subset of cDNA's with complementary sequence—either adjacent to the adaptor for one strand or adjacent to the polyA for the other strand. For a Type IIS enzyme, adaptors are employed that will ligate with the possible different cohesive ends generated when the enzyme cuts the double-stranded DNA. Thus a population of adaptors may be employed to be complementary to all possible cohesive ends within the population of DNA after cutting/digestion by the Type IIS enzyme. Primers are used in the PCR that anneal with the adaptors.

Primers may be labelled, and the labels may correspond to the relevant A, T, C or G nucleotide at a corresponding position in the relevant primer variable region. This means that double-stranded DNA produced in the PCR is labelled, and that the combination of the label and the length of the product DNA provides a characteristic signal. Otherwise, the combination of length of the product and (i) PCR primer used for a Type II enzyme digest or (ii) adaptor used for a Type IIS digest, provides a characteristic signal.

A given gene in a sample will when cut by a given restriction enzyme and amplified using an adaptor that anneals in accordance with the method produce a fragment that will give rise to a signal that is a composed of the length and sequence information. This may not be directly uniquely assignable by a simple look-up to a single gene in the database, since multiple genes may happen to give rise to the same fragment signal. However, by use of two or more different restriction enzymes to generate different populations of fragments for the same sample, multiple signals can be obtained allowing for unique identification of a fragment. Thus for the same sample treated with different restriction enzymes, different patterns of signals are generated and this allows the patterns to be compared to a database of signals for known mRNAs using a combinatorial identification algorithm.

Patterns of signals generated for a sample using two or more different restriction enzymes may be compared with a pattern generated from a database of known sequences assigned as “virtual genes”, wherein possible polyA sites are represented. A virtual gene is defined as representing a possible polyadenylation site downstream of a stop codon within an actual gene, and the virtual genes in the database may collectively represent some or all possible polyadenylation sites within one or more actual genes, or may represent a subset of candidate or potential polyadenylation sites determined by any suitable means, for example computational analysis and/or experimentation. Virtual genes may be included for sites within a few bases around an experimentally determined polyA site (e.g. to allow for some experimental error) or around a predicted polyA site. Virtual genes may be included for any one or more potential sites downstream of any plausible polyA signal computationally determined. In a preferred embodiment, a combination of available annotation, e.g. by virtue of computationally determined polyA signals and/or experimental evidence, is combined. Each annotated position may be given a score, with scores also being given to intervening positions according to the distance from an annotated position. Application of a threshold set allows for a reduction in the level of false positives and false negatives. In other embodiments all potential sites may be used, e.g. for analysis of yeast or mouse genes.

Virtual genes may be included for possible polyA sites within for example 5-10 bases for an experimentally determined polyA site, or 10-20 for a computationally predicted polyA site, depending on the likelihood of the polyA site being correct. Preferably a system of scoring is employed, wherein experimentally determined polyA sites are given higher scores than those predicted computationally, and potential sites around the determined or predicted sites are given falling scores, with the scores falling more quickly for experimentally determined polyA sites. Use of a threshold value for the score reduces the number of virtual genes to be employed in the database. Thus, for example, virtual genes may in one embodiment be included in the database for experimentally determined polyA sites wherein virtual genes are included for each site within 5, 6, 7, 8, 9 or 10 nucleotides of the experimentally determined polyA sites. Virtual genes may in one embodiment be included in the database for predicted polyA sites within 5, 6, 7, 8, 9, 10, 11, 12, 13, 14, 15, 16, 17, 18, 19 or 20 nucleotides of the predicted polyA sites.

A virtual gene that corresponds with a fragment that appears in the results of multiple digest reactions is thus identified as real.

In accordance with embodiments of the present invention, such technology may be employed as follows:

1. All the genes in the database which correspond to a fragment are listed. This forms a list of possibly expressed genes for each experiment.

2. Then the genes which definitely do not correspond to a fragment are listed (i.e. those which should give a fragment of a length and/or partial sequence which was not found in the experiment). This forms a list of definitely unexpressed genes for each experiment.

3. The unexpressed genes in each experiment are then removed from the list of possibly expressed genes in each other experiment.

4. The result is a list for each experiment where in most cases each fragment retains a single candidate gene identification.

This works, because each real gene actually present in the sample should be seen “k” times, where “k” is the number of experiments i.e. number of different restriction digests performed, with “k” fragments. If then less than the number k fragments are seen for a virtual gene in the database, then that virtual gene was not actually present as a real gene in the sample, provided of course that the gene is capable of being cut by all of the different restriction enzymes used in the experiments, i.e. the gene includes the appropriate restriction enzyme recognition site. Where, of the k different restriction enzymes, a gene does is not cut by a number of enzymes “λ”, then the gene should give rise to “k−λ” fragments. The gene can still be eliminated if fewer fragments than k−λ are seen. Thus, for example, if a gene is subject to three digests (k=3), but can only be cut by 2 of those (λ=1), then a virtual gene candidate can still be eliminated if only 1 fragment is observed instead of the expected 2.

Thus, resolving the combinatorial equations for signals generated for fragments generated from actual genes, using actual polyA sites, present in the sample compared with virtual genes in the database representative of all hypothetically possible polyA sites, allows for identification of the actual polyA sites employed in the genes actually present in the sample.

The analysis may be performed quantitatively, e.g. as described in GB0018016.6 and PCT/IB01/01539, if an abundance measure is available for each fragment (e.g. peak height in an electrophoresis trace):

1. All the genes in the database which correspond to a fragment in each experiment are listed (i.e. those virtual genes that match the signal for length and/or sequence information generated for the fragments produced from the actual genes in the sample). This forms a list of possibly expressed genes for each experiment (i.e. those virtual genes that may be real and actually be present in the sample). For each fragment in each experiment an equation is written of the form Fi=m1+m2+m3, where 1, 2, 3 etc are the id's of the genes and Fi is the intensity of the signal from the fragment. Each virtual gene which may correspond to a fragment peak in the electrophoresis appears as a term on the right-hand side.

2. For example, if a peak at 162 bp corresponds to virtual genes 234, 647 and 78 in the database, and it has intensity 2546, then the corresponding equation is written as

2546=m234+m647+m78

3. Then for each experiment, the virtual genes which definitely do not correspond to a fragment are listed (i.e. those which if present in the sample would give a fragment of a length which was not found in the experiment). This forms a list of definitely unexpressed genes for each experiment, i.e. virtual genes that are definitely not actually in the sample. For each virtual gene on that list, an equation is written of the form:

0=m657

where 657 is the virtual gene id, as above.

4. A system of simultaneous equations is thus obtained with m (=the number of genes in the sample) unknowns and n km equations (where k is the number of experiments). If all genes run as singlets in all experiments then n=km because each gene will appear just once in its own equation. The more they run as doublets or multiplets the smaller n will be. As long as n>m, however, the system is over-determined and can thus be solved using standard numerical methods to find a least-squares solution. For example, the backslash operator in the standard numerical an analysis package MATLAB (The MathWorks, Inc.) can be used.

5. The least-squares solution of the system gives for each gene the best approximation of its expression level. The more experiments that are performed, the better the approximation will be. Errors can be estimated by computing residuals (that is, by inserting the estimated gene activities in the equations to obtain calculated peak intensities and comparing those to the measured intensities). Simulations show that a system of 100 000 equations in 50 000 unknowns can be solved in 16 hours on a regular PC.

The present invention is a novel approach to finding polyadenylation sites. By extension, it can also be applied to mapping any functional site that would generate a difference in the length of nucleic acid fragments after restriction enzyme cleavage. Such sites include the restriction enzyme sites themselves, alternative splicing of RNA and 5′ capping sites. All that is required is to generate additional virtual genes representing the theoretical possibilities, e.g. representing combinations of possible restriction sites for a particular enzyme andd possible polyA sites. It is thus a novel general method for the systematic discovery of functional gene features on a global scale.

In brief, a method according to the invention may involve generating a dataset containing length and partial sequence information for a large number of fragments obtained from nucleic acid in a sample, and then using a combinatorial identification algorithm to assign gene sequences in a database to fragments in such a way that alternative polyadenylation can be determined.

The dataset is redundant, i.e. each gene to be analyzed is represented multiple times in the dataset. Examples of such datasets include those generated in accordance with the profiling method of GB0018016.6 and PCT/IB01/01539, and as disclosed herein, in which an mRNA sample is converted to cDNA, subjected to restriction with enzymes, preferably type IIS enzymes, followed by adaptor ligation in multiple subreactions (e.g. 256 where the restriction enzyme used cuts with a four base overhang, such as FokI) and PCR amplification. Each such profile carries information about the length and a number of basepairs of sequence for each fragment (e.g. 9 basepairs). If the dataset includes a number of such profiles, that number being two or more, or three or more, e.g. two or three or four, preferably three, generated with different enzymes, then each gene in the sample will be represented that same number of times by different fragments.

Given a dataset of the required composition, one may then use a combinatorial identification algorithm to assign candidate genes from a sequence database.

For discovery of polyadenylation sites or determining polyA site usage in a sample, assignment criteria are employed wherein each potential polyadenylation site is considered as an independent candidate gene (a “virtual gene”). With the dataset generated from the restriction digests containing sufficient redundancy of information, it can be unambiguously determined which of all possible candidates including the virtual genes, was actually present in the sample. This simultaneously provides direct experimental evidence for the presence of an alternative polyadenylation site for all confirmed virtual genes.

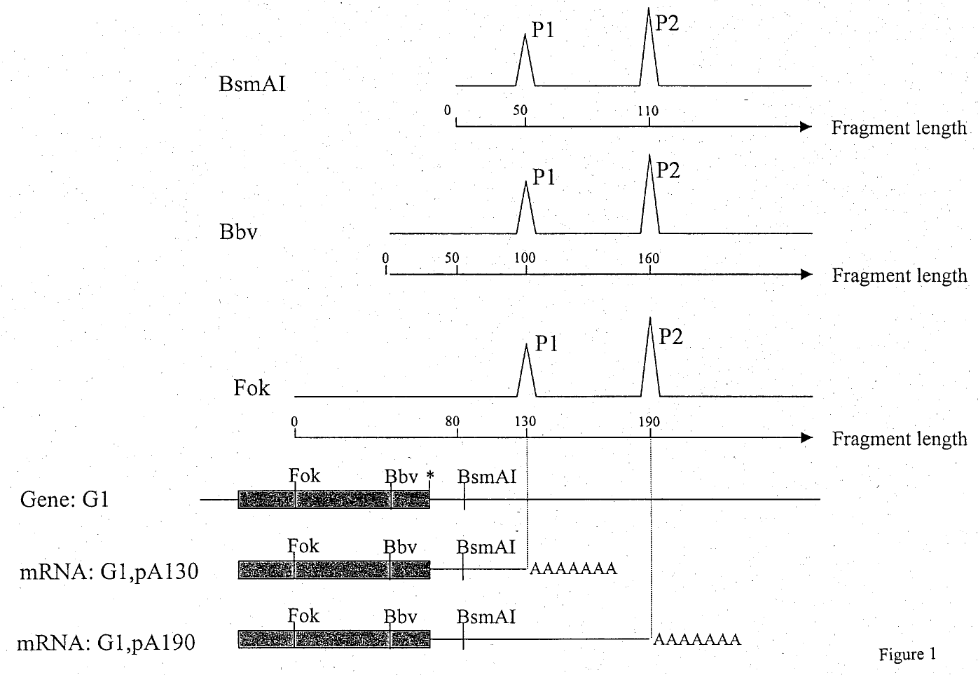

FIG. 1 illustrates an embodiment of the present invention involving discovery of polyadenylation sites. Given a gene with two candidate poly(A) sites, and given three gene profiles produced in this case by restriction enzyme cleavage with three different enzymes, the appearance of peaks corresponding to the candidate poly(A) sites provides direct experimental evidence for their existence. Note that a change in the position of a poly(A) site affects the fragments coming from that site in all three profiles. By implication, it is evident that the more information can be obtained about each gene (i.e. the more independent profiles are produced), the more confident one can be about each poly(A) site discovered. Conversely, the more information can be obtained about each gene, the more candidate poly(A) sites can be introduced and resolved.

The present invention can be used to discover alternative polyadenylation sites in a sample of expressed genes, or determine which of alternative polyadneylation sites are present. Because alternative polyadenylation often has been selected during evolution to confer tissue-specific regulation of mRNA turnover, their discovery and identification in a straightforward fashion and on large scale, as embodiments of the present invention allow, is an important contribution to the art.

According to one aspect of the present invention there is provided a method for determining the presence of and/or identifying a polyadenylation site or alternative polyadenylation sites within a sequence of a transcribed gene or sequences of transcribed gene variants present or potentially present in a sample, the method comprising:

(a) generating a dataset comprising a set of signals obtained for individual gene fragments within a population of gene fragments produced from transcribed genes in the sample, wherein the signal for an individual gene fragment comprises a combination of length and partial sequence information and a magnitude component for that gene fragment, wherein the dataset contains a magnitude component of zero for combinations of length and partial sequence information determined not to be present in the population and the magnitude component of the signal for gene fragments for which the combination of length and partial sequence information is determined to be present is either qualitative to indicate presence in the population of a gene fragment with that combination or quantitative to provide an indication of the amount of individual gene fragments present in the population; and

(b) assigning to gene fragments one or more gene candidates within a database by comparing signals within the dataset with the database, the database comprising data representing mRNA's with known polyA sites and/or “virtual genes”, wherein virtual genes are defined as each representing a possible polyadenylation site within an actual gene,

(c) eliminating from results gene candidates which are each assigned to at least one signal of magnitude zero,

(d) thereby obtaining results defining a set of one or more genes or gene variants each being a mRNA with a known polyadenylation site and/or virtual gene assigned to a signal with non-zero magnitude in the dataset, which results provide indication of actual presence of said set of one or more genes or gene variants in said sample.

The virtual genes in the database may be provided by scoring possible polyadenylation sites within an actual gene for likelihood of actual occurrence and including in the database virtual genes that exceed a defined threshold of likelihood of actual occurrence.

The virtual genes in the database may collectively represent all possible polyadenylation sites within one or more actual genes.

A population of gene fragments may be provided by cutting cDNA copies of mRNA in a sample and purifying cut gene fragments that each comprise a terminal polyA sequence.

A population of gene fragments may be provided by digesting with a restriction enzyme cDNA copies of mRNA in a sample and purifying digested gene fragments that each comprise a terminal polyA sequence.

An embodiment of the method comprises:

providing a first population of gene fragments by digesting with a first restriction enzyme cDNA copies of mRNA in a sample and purifying digested gene fragments that each comprise a terminal polyA sequence; and

providing a second population of gene fragments by digesting with a second restriction enzyme cDNA copies of mRNA in the sample and purifying digested gene fragments that each comprise a terminal polyA sequence; and optionally

providing a third population or further populations of gene fragments by digesting with a third restriction enzyme, or further restriction enzymes, cDNA copies of mRNA in the sample and purifying digested gene fragments that each comprise a terminal polyA sequence.

A method of the invention wherein first and second populations are provided, and optionally a third population or further populations, may comprise:

determining the identity of one or more mRNA's with known polyA sites and/or virtual genes with a non-zero magnitude signal within signals for each of the first population and the second population, and optionally the third population or the further populations, within the dataset, whereby a mRNA with known polyA site and/or virtual gene that has a non-zero magnitude signal within the signals for both the first and second populations or all the populations is identified as corresponding to a polyadenylation site in a transcribed gene or transcribed gene variants present in the sample.

In preferred embodiments, three different restriction enzymes are employed, providing three populations of gene fragments.

The signal generated for a gene fragment in a population may be quantitatively related to the amount of the mRNA in the sample by means of including in provision of the signal quantitative determination of the amount of gene fragment of the defined length and sequence information. The amount of gene fragment is generally measured after amplification, but can be related back to the amount of corresponding mRNA in the sample (in other words the expression level).

A restriction enzyme employed in preferred embodiments may cut double-stranded DNA with a frequency of cutting of 1/256-1/4096 bp, preferably 1/512 or 1/1024 bp.

Where the restriction enzyme is a Type II restriction enzyme, it is preferred to use HaeII, ApoI, XhoII or Hsp 921. Where the restriction enzyme is a Type IIS restriction enzyme, it is preferred to use FokI, BbvI or Alw261. Other suitable enzymes are identified by REBASE (rebase.neb.com or find REBASE using any web browser).

Preferably, the restriction enzyme digests double-stranded DNA to provide a cohesive end of 2-4 nucleotides. For a Type IIS restriction enzyme a cohesive end of 4 nucleotides is preferred.

As discussed, information is obtained by generating two or more patterns of signals for gene fragments derived from the sample using a second, or second and third, or further different Type II or Type IIS restriction enzyme or enzymes. In some preferred embodiments of the present invention, three different restriction enzymes are used.

The signal for a gene fragment may comprise quantitative information on amount of the gene fragment present.

A method in accordance with embodiments of the present invention may comprise:

synthesizing a cDNA strand complementary to each mRNA in the sample using the mRNA as template, thereby providing a population of first cDNA strands;

removing the mRNA;

synthesizing a second cDNA strand complementary to each first strand, thereby providing a population of double-stranded cDNA molecules;

digesting the double-stranded cDNA molecules with a Type II or Type IIS restriction enzyme to provide a population of digested double-stranded cDNA molecules, each digested double-stranded cDNA molecule having a cohesive end provided by the restriction enzyme digestion;

ligating a population of adaptor oligonucleotides to the cohesive end of each of the digested double-stranded cDNA molecules, the adaptor oligonucleotides each comprising an end sequence complementary to a cohesive end and a primer annealing sequence, thereby providing double-stranded template cDNA molecules each comprising a first strand and a second strand wherein the first strand of the double-stranded template cDNA molecules each comprise a 3′ terminal adaptor oligonucleotide and the second strand of the double-stranded template cDNA molecules each comprise a 3′ terminal polyA sequence;

purifying said double-stranded template cDNA molecules;

performing polymerase chain reaction amplification on the double-stranded template cDNA molecules having a sequence complementary to a 3′ end of an mRNA using a population of first primers and a population of second primers,

wherein the first primers each comprise a sequence which anneals to a primer annealing sequence of an adaptor oligonucleotide; and

where the restriction enzyme is a Type II enzyme the first primers each comprise at least one 3′ terminal variable nucleotide and optionally more than one 3′ terminal variable nucleotides wherein the variable nucleotide is, or at a corresponding position within the variable nucleotides each first primer has, a nucleotide selected from A, T, C and G, whereby the population of first primers primes synthesis in the polymerase chain reaction of first strand product DNA molecules each of which is complementary to the first strand of a template cDNA molecule that comprises adjacent to the primer annealing sequence within the first strand of the template cDNA molecule a nucleotide or sequence of nucleotides complementary to the variable nucleotide or nucleotides of a first primer within the population of first primers; or

where the restriction enzyme is a Type IIS enzyme the first primers prime synthesis in the polymerase chain reaction of first strand product DNA molecules each of which is complementary to the first strand of a template cDNA molecule that comprises within the first strand of the template cDNA molecule a sequence of nucleotides complementary to an end sequence of an adaptor oligonucleotide in the population of adaptor oligonucleotides;

the second primers comprise an oligoT sequence and a 3′ variable portion conforming to the following formula: (G/C/A) (X)ₙ wherein X is any nucleotide, n is zero, at least one or more than one (e.g. two); whereby the population of second primers primes synthesis in the polymerase chain reaction of second strand product DNA molecules each of which is complementary to the second strand of a template cDNA molecule that comprises adjacent to polyA within the second strand of the template cDNA molecule a nucleotide or nucleotides complementary to the variable portion of a second primer within the population of second primers;

whereby the polymerase chain reaction amplification provides a population of double-stranded product DNA molecules (said gene fragments) each of which comprises a first strand product DNA molecule and a second strand product DNA molecule;

separating double-stranded product DNA molecules on the basis of length; and

detecting said double-stranded product DNA molecules;

whereby a signal for each double-stranded product DNA molecule is provided by combination of length of said double-stranded product DNA molecules and (i) first primer variable nucleotide or nucleotides, where a Type II restriction enzyme is employed, or (ii) adaptor oligonucleotide end sequence, where a Type IIS restriction enzyme is employed;

wherein signals are provided for first and second populations and optionally a third population or further populations of double-stranded product DNA molecules (said gene fragments) obtained by means of first and second different restriction enzymes and optionally a third different restriction enzyme or further different restriction enzymes.

Removing mRNA from the first strand may be by any approach available in the art. This may involve for example digestion with an RNase, which may be partial digestion, and/or displacement of the mRNA by the DNA polymerase synthesizing the second cDNA strand (as for example in the Clontech™ SMART™ system).

In embodiments of the present invention, signals in the dataset may be compared with a database of signals determined or predicted for mRNA's with known polyA sites and/or said virtual genes, by:

(i) listing all mRNA's with known polyA sites and/or virtual genes in the database which may correspond to a gene fragment in each of said first and second and optionally third or further populations, forming a list of mRNA's with known polyA sites and/or virtual genes possibly present for each population, and

(ii) listing mRNA's with known polyA sites and/or virtual genes which definitely do not correspond to a gene fragment, forming a list of mRNA's with known polyA sites and/or virtual genes definitely not present for each population, then

(iii) removing the mRNA's with known polyA sites and/or virtual genes definitely not present from the list of mRNA's with known polyA sites and/or virtual genes possibly present for each population, and

(iv) generating a list of mRNA's with known polyA sites and/or virtual genes possibly present and mRNA molecules definitely not present by combining each list generated for each population in (iii);

thereby identifying one or more mRNA's with known polyA sites and/or virtual genes as corresponding to mRNA actually present in the sample.

This may involve:

(i) listing all mRNA's of known polyA site and/or virtual gene in the database which may correspond to a gene fragment in each of the first and second and optionally third or further populations, and forming a set of equations of the form Fi=m₁+m₂+m₃, wherein Fi is the intensity of the signal from the fragment, the numerals are the identity of the mRNA's of known polyA sites and/or virtual genes in the database and wherein each mRNA with known polyA site or virtual gene which may correspond to a gene fragment appears as a term on the right-hand side;:

(ii) for each experiment listing mRNA's of known polyA site and/or virtual genes which definitely do not correspond to a gene fragment in each population, and writing for each mRNA of known polyA site and/or virtual gene which definitely does not correspond to a gene fragment in each population an equation of the form 0=m₄, wherein the numeral is the identity of the mRNA of known polyA site and/or virtual gene in the database;

(iii) combining the sets of equations to form a system of simultaneous equations wherein the number of equations is greater than the number of transcribed genes or transcribed gene variants present or potentially present in the sample;

(iv) determining an amount of the expression level of each transcribed gene or transcribed gene variant by solving the system of simultaneous equations; and

(v) including the determined amounts of the expression levels within the signals provided for each gene fragment.

First primers employed in embodiments of the present invention may each have one variable nucleotide; in other embodiments they may each have two variable nucleotides, each of which may be A, T, C or G; in other embodiments they may each have three variable nucleotides, each of which may be A, T, C or G.

Each first primer may be labelled with a label to indicate which of A, T, C and G is said variable nucleotide or is present at said corresponding position within the variable nucleotides of the first primer.

Adaptor oligonucleotides in the population of adaptor oligonucleotides may be ligated to cohesive ends of digested double-stranded cDNA molecules in separate reaction vessels from different adaptor oligonucleotides with different end sequences.

In embodiments of methods of the present invention each reaction vessel may contain a single adaptor oligonucleotide end sequence; in other embodiments each reaction vessel may contain multiple adaptor oligonucleotide end sequences, each adaptor oligonucleotide sequence in a reaction vessel comprising a different end sequence and primer annealing sequence from the end sequence and primer annealing sequence of other adaptor oligonucleotide sequences in the same reaction vessel, corresponding multiple first primers being employed in the polymerase chain reaction amplification in each reaction vessel.

In each first primer used for PCR following digestion with a Type II enzyme, there may be a single variable nucleotide, or a variable nucleotide sequence of more than one nucleotide, e.g. two or three. At each position in a variable sequence, first primers may be provided such that each of A, C, G and T is represented in the population.

In each second primer (comprising oligo dT), n may be 0, 1 or 2.

No variable nucleotide is need in the primers used for PCR where a Type IIS restriction enzyme is employed because variability in the adaptor sequence is provided by the cohesive end. Generally, where a Type IIS restriction enzyme is employed a population of adaptors is provided such that all possible cohesive ends for the restriction enzyme are represented in the population, and each adaptor may be ligated to a fraction of the sample in a separate reaction vessel. The adaptor used in each reaction vessel will then be known and combination of this information with the length of double-stranded product DNA molecules provides the desired characteristic pattern.

In a preferred embodiment, when ligating adaptors, the adaptors may be blocked on one strand, e.g., chemically. This may be achieved using a blocking group such as a 3′ deoxy oligonucleotide, or a 5′ oligonucleotide in which the phosphate group has been replace by nitrogen, hydroxyl or another blocking moiety. This allows ligation at the other, unblocked strand and can be used to improve specificity. A specificity greater than 250:1 can be obtained. PCR can proceed from the single ligated strand. In addition, ligation conditions have been identified which improve ligation specificity and/or efficiency, as described in the materials and methods. It has been found that these conditions are advantageous in achieving specificity in the ligation of adaptors with up to four variable base pairs.

For convenience, multiple adaptors may be combined in a single reaction vessel, in which case each different adaptor in a given vessel (with a different end sequence complementary to a cohesive end within the population of possible cohesive ends provided by the Type IIS restriction enzyme digestion) comprises a different primer annealing sequence. For instance three different adaptors may be combined in one reaction vessel. Corresponding first primers are then employed, and these may be labelled to distinguish between products arising from the respective different adaptor oligonucleotides.

Where a Type II enzyme is used, the first primers may be labelled, although where individual polymerase chain reaction amplifications are performed in separate reaction vessels there is already knowledge of which first primer is used. Otherwise, labelling provides convenient information on which first primer sequence is providing which double-stranded DNA product molecule.

Conveniently, three different first primer PCR amplifications can be performed in each reaction vessel, with each first primer being labelled appropriately (optionally with employment of a labelled size marker).

Separation may employ capillary or gel electrophoresis. A single label may be employed per reaction, with four dyes per capillary or lane, one of which may carry a size marker.

Labels may conveniently be fluorescent dyes, allowing for the relevant signals (e.g. on a gel) following electrophoresis to separate double-stranded product DNA molecules on the basis of their length to be read using a normal sequencing machine.

Populations of gene fragments generated to provide the signals of the dataset for comparison with the database can be prepared on a solid support, where each transcribed gene or transcribed gene variant in the sample is represented by a unique gene fragment. The populations can be displayed on a capillary electrophoresis machine after PCR amplification with fluorescent primers. In order to reduce the number of bands in each electropherogram, the initial library may be subdivided, e.g. using one of the following two methods (α) and (β).

(α) For libraries generated with an ordinary Type II enzyme, an adapter is ligated to the cohesive end of each fragment. The adaptor comprises a portion complementary to the cohesive end generated by the restriction enzyme and a portion to which a primer anneals. One primer annealing sequence may be used, or a small number, e.g. 2 or 3, of different sequences showing minimal cross-hybridisation, to allow that small number of independent reactions to proceed in a single reaction vessel. The library is then split into a number of different reaction vessels and a subset of the fragments in each vessel is PCR amplified using primers compatible with the 3′ (oligo-T) and 5′ (universal adapter) ends carrying a few extra bases protruding into unknown sequence. Thus in each reaction a different combination of protruding bases causes selective amplification of a subset of the fragments.

(β) For libraries generated by Type IIS enzymes—which cleave outside their recognition sequence giving a gene-specific cohesive end—the library is split into a number of different reaction vessels. A set of adapters is designed containing a universal invariant part and a variable cohesive end such that all possible cohesive ends are represented in the set. In each reaction vessel a single such adapter is ligated. The subset of fragments in each vessel carrying adapters is then amplified with universal high-stringency primers.

In both methods, the resulting reactions may be run separately on a capillary electrophoresis machine which quantifies the fragment length and abundance, indicating the relative abundances of the corresponding mRNAs in the original sample.

For each gene fragment, the following are known and are used to provide the characteristic signal:

the restriction enzyme site used to generate the gene fragments (e.g. 4-8 bases);

its length (representative of the distance between the restriction enzyme cutting site and the polyA site);

sub-reaction (given by the subdivision method, but generally corresponding to an additional 4-6 bases).

Enough information is generated to identify each fragment with known sequences from a database. This may be performed by selecting a combination of fragment length distribution (given by the enzyme) and subdivision (given by the protruding bases and/or by the cohesive end (Type IIS)). As few as two bases (16 sub-reactions) or as many as 8 (65536 sub-reactions) can be used; if a small transcriptome is being analyzed, a small number of sub-reactions may be enough; if a high-throughput analysis method is available a large number of sub-reaction allows the separation of very large numbers of genes or gene variants. In practice, between four and six bases are usually used.

Experimental Exemplification

Ligation of multiple adapters to cohesive ends generated by a Type IIS enzyme to generate subsets (frames), followed by PCR with universal primers. Discovery of alternative polyadenylation sites by combinatorial identification.

An experiment was performed on mouse mRNA as follows. Further details of the materials and methods are included below.

cDNA was synthezised on a solid support. The first strand was synthesized by reverse transcriptase (RT) from mRNA primed with biotinylated oligo-dT. The second strand was produced by an RNase, which cleaves the mRNA, and a DNA Polymerase, which primes off small RNA fragments which are left by the RNase, displacing other RNA fragments as it goes along. The double stranded cDNA was attached to streptavidin-coated Dynabeads (Dynal, Norway).

The cDNA was then cleaved with a class-IIS endonuclease with a recognition sequence of 5 nucleotides. Class IIS restriction endonucleases cleave double-stranded DNA at precise distances from their recognition sequences (at 9 and 13 nucleotides from the recognition sequence in the example of the class IIS restriction endonuclease FokI). Other examples of class IIS restriction endonucleases include BbvI, SfaNI and Alw26I and others described in Szybalski et al. (1991) Gene, 100, 13-26. The 3′ parts of the cDNA attached to the solid support were then purified using the solid support. The cDNA was then divided into 256 fractions and a different adaptor was ligated to the fragments in each fraction.

One enzyme used was FokI. FokI cleavage leads to four nucleotides 5′ overhang, with each overhang consisting of a gene-specific but arbitrary combination of bases. One adaptor carrying a single possible nucleotide combination in these four positions was used in each fraction i.e. a total of 256 adapters and fractions. The adaptors were blocked on one strand, improving specificity by forcing ligation to occur on the other strand only. Again by means of the solid support, the cDNA was then purified to remove excess non-ligated adaptor. PCR was performed on the 256 fractions using one universal primer complementary to the constant part of the adapter sequence and one complementary to the poly-A tail.

The 3′ primers were oligo dT and therefore complementary to the polyadenylation sequence of the original mRNA. Each primer was designed with a base extending into unknown sequence, guanine, adenosine or cytosine. (A second or still further base may be included, being any of guanine, adenosine, thymine or cytosine.) Each well received a mixture of the three possible 3′ primers. This ensured that the 3′ primer always directed the polymerase to the beginning of the poly-A tail, giving a defined and reproducible fragment length.

The resulting PCR products were purified and loaded onto an ABI prism capillary sequencer. The PCR fragments representing the expressed genes were thus separated according to size and the fluorescence of each fragment quantified using the detector and software supplied with the capillary electrophoresis equipment.

This procedure was performed three times with different Type IIS restriction enzymes (FokI, BbvI and BsmAI) so that three independent profiles were obtained for the same sample. Combining the information unique to each fragment in this analysis, i.e. 9 nucleotides (including the FokI recognition sequence and cleavage site) and the size from polyadenylation to the FokI restriction site obtained from the capillary sequencer, the identity (EST, gene or mRNA identity) of each mRNA can be established using combinatorial algorithms as set out herein (see also GB0018016.6 and PCT/IB01/01539).

A simulated dataset was constructed, corresponding to expression of 5247 genes from the mouse genome. 3094 known polyadenylation sites were used, and 11057 polyadenylation sites were randomly defined, but not made accessible in the gene database, in a 10 nucleotides neighbourhood of known polyadenylation sites, or in a 10-30 nucleotide region 3′ to putative and known polyadenylation signals.

When the simulted dataset was analyzed using the algorithm as set out herein with the original mouse gene database, not containing information on the 11057 defined additional polyadenylation sites, it correctly assigned expression to 5226 of the expressed genes and 3004 out of the 3094 known active polyadenylation sites.

Most importantly, it located 10438 of the 11057 non-registered (“unknown” in the experiment) polyadenylation sites, proving usefulness of the present invention for detecting alternative polyadenylation sites.

Use of PCR primers with one or more bases protruding into unknown sequence to generate subsets (frames) for generating signals for gene fragments corresponding to transcribed mRNA in a sample.

RNA was purified from a sample according to standard techniques. The RNA was denatured at 65° C. for 10 minutes and added to Oligotex beads (Qiagen) and annealed to the oligo dT template covalently bound to the beads. A first strand cDNA synthesis was carried out-using the mRNA attached to the Oligotex beads as template. This first strand cDNA therefore becomes covalently attached to the Oligotex beads (Hara et al. (1991) Nucleic Acids Res. 19, 7097). Second strand synthesis was performed as described in Hara et al above. Briefly, the first strand was synthesized by reverse transcriptase (RT) from mRNA primed with oligo-dT. The second strand was produced by an RNase, which cleaves the mRNA, and a DNA Polymerase, which primes off small RNA fragments which are left by the RNase, displacing other RNA fragments as it goes along. The double-stranded cDNA attached to the Oligotex beads was purified and restriction digested with HaeII. HaeII was used. Alternative enzymes include ApoI, XjoII and Hsp921 (Type II) and FokI, BbvI and Alw261 (Type IIS). The cDNA was again purified retaining the fraction of cDNA attached to the Oligotex.

An adaptor was ligated to the HaeII site of the cDNA. The adaptor contained sequences complementary to the HaeII site and extra nucleotides to provide a universal template for PCR of all cDNAs. The cDNA was then again purified to remove salt, protein and unligated adaptors.

The cDNA was divided into 96 equal pools in a 96 well dish. In order to PCR amplify only a subset of the purified fragments in each well, a multiplex PCR was designed as follows.

The 5′ primers were complementary to the universal template but extended two bases into the unknown sequence. The first of these bases was either thymine or cytosine, corresponding to a wobbling base in the HaeII site, while the second was any of guanine, cytosine, thymine or adenosine. Each 5′ primer was fluorescently coupled by a carbon spacer to fluorochromes detectable by the ABI Prism capillary sequencer. The fluorochrome was matched to the second base. Each well received four primers with all four fluorochromes (and hence all four second bases); half of the wells received primers with a thymine first base, half with a cytosine first base.

The 3′ primers were oligo dT and therefore complementary to the polyadenylation sequence of the original mRNA. Each primer was designed with three bases extending into unknown sequence, the first of which was either guanine, adenosine or cytosine, while the other two was any of the four bases. Each well received a single 3′ primer. Thus, the PCR reaction was multiplexed into 384 sub-reactions: 96 wells with four fluorochrome channels in each.

A standard PCR reaction mix was added, including buffer, nucleotides, polymerase. The PCR was run on a Peltier thermal cycler (PTC-200). Each primer pair used in this experiment recognises and amplifies only genes containing the unique 4 nucleotide combination of that primer pair.

The size of the PCR fragment of each of these genes corresponds to the length between the polyadenylation and the closest HaeII site.

The resulting PCR products were isopropanol precipitated and loaded onto an ABI prism capillary sequencer. The PCR fragments representing the expressed genes were thus, separated according to size and the fluorescence of each fragment quantitated using the detector and software supplied with the ABI Prism.

The combination of primers used lead to a theoretical mean of ˜70 PCR products in each fluorescent channel and sample (based on 20% genes expressed in a given sample and a total of 140,000 genes). Analysis of statistical size distribution of 3′ fragments including the polyadenylation generated from known genes following HaeII restriction digestion, showed that an estimated 80% can be uniquely identified based on frame and length of fragment alone. The ABI prism has 0.5% resolution between 1-2,000 nucleotides. Allowing for this uncertainty, ˜60% of the expressed genes can be uniquely identified. Using an additional parallel experiment using the same protocol but replacing the HaeII enzyme with another 5 base cutting restriction enzyme increases the theoretical limit to ˜96% and the practical limit (given the resolution of the ABI Prism) to ˜85% of all transcripts in the genome.

The level of each mRNA in the sample corresponds to the signal strength in the ABI prism. Combining the information unique to each fragment in this analysis, i.e. 8.5 nucleotides (including the HaeII recognition sequence) and the size from poly adenylation to the HaeII restriction site, the identity of each mRNA can thus be established by comparison with a database containing mRNA's of known polyA sites and/or virtual genes which represent all theoretically possible polyA sites downstream of the stop codon in one or more mRNA's.

A searchable database on all known genes and unigene EST clusters was constructed as follows.

Unigene, a public database containing clusters of partially homologous fragments was downloaded (although the invention may be used with any set of single or clustered fragments). For each cluster, all fragments containing a polyA signal and a polyA sequence were scanned for an upstream HaeII site. If no HaeII site was found, then the fragments were extended towards 5′ using sequences from the same cluster until a HaeII site was found. Then, the frame was determined from the base pairs adjacent to the HaeII and the polyA sequences and the length of a HaeII digest was calculated. The frame and length were used as indexes in the database for quick retrieval.

The output from the ABI Prism was run against the database, thus allowing the identification of expression level of any one or more of the known genes and ESTs actually expressed in the RNA contained in the sample of this study.

Ligation of multiple adapters to cohesive ends generated by a Type XIS enzyme to generate subsets (frames), followed by PCR with universal primers.

In another set of experiments the method was simplified and an increased resolution was achieved. cDNA was synthezised on solid support as described in the preceding section, but this time using magnetic DynaBeads(as described in Materials and Methods). The cDNA was then cleaved with a class-IIS endonuclease with a recognition sequence of 4 or 5 nucleotides.

Class IIS restriction endonucleases cleave double-stranded DNA at precise distances from their recognition sequences (at 9 and 13 nucleotides from the recognition sequence in the example of the class IIS restriction endonuclease FokI). Other examples of class IIS restriction endonucleases include BbvI, SfaNI and Alw26I and others described in Szybalski et al. (1991) Gene, 100, 13-26. The 3′ parts of the cDNA were then purified using the solid support as described above. The cDNA was then divided into 256 fractions and a different adaptor was ligated to the fragments in each fraction.

For example, FokI cleavage leads to four nucleotides 5′ overhang, with each overhang consisting of a gene-specific but arbitrary combination of bases. One adaptor carrying a single possible nucleotide combination in these four positions was used in each fraction i.e. a total of 256 adapters and fractions.

Highly specific ligation of adaptors bearing a given nucleotide combination to the complementary nucleotide sequence in the fragment population was achieved by chemically blocking the adaptors on one strand, by using a deoxy oligonucleotide. As a result, ligation was forced to occur only on the other strand.

The specificity of ligation was tested using a single template, bearing a four base pair overhang. Adaptors were designed which were either exactly complementary to this overhang, or which had 1, 2 or 3 mismatches. Adaptors were ligated to the template, PCR was performed, and the relative amount of product obtained from each of the adaptor sequences was assessed.

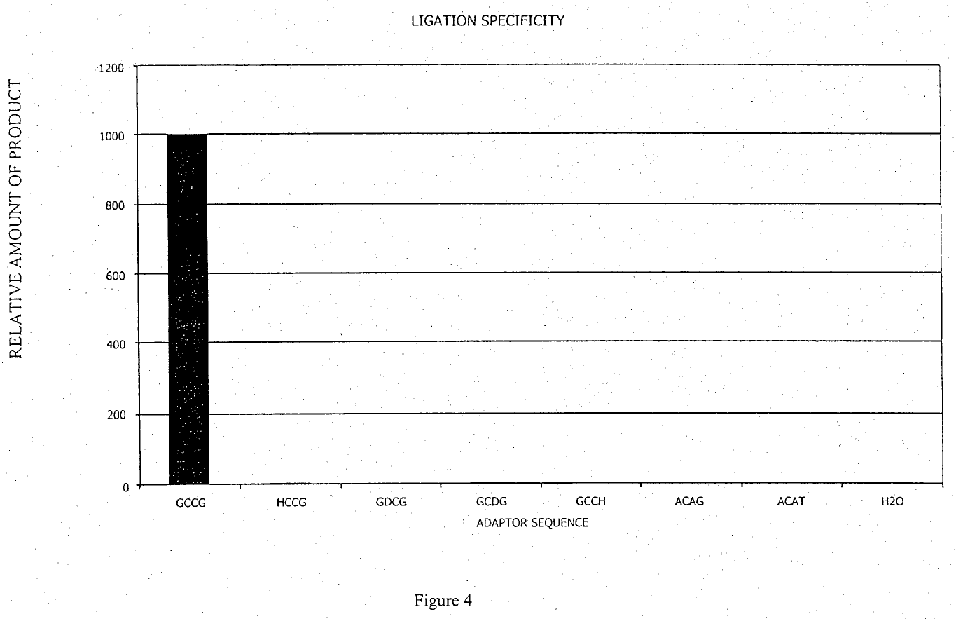

It was found that high specificity was achieved for an adaptor blocked by including a deoxy nucleotide at the 3′ end of the upper strand (and also at the 3′ end of the lower strand in order to prevent interference at the PCR step). The results are shown in FIG. 4. The sequence GCCG is exactly complementary to the sequence of the template oligonucleotide. It can be seen that the amount of product bearing this sequence is approximately 250 times greater than the amount of product bearing sequences with one or more mismatches. Hence it can be seen that the ligation reaction proceeds with high specificity.

Adaptors which were chemically blocked by introducing at the 5′ end of the lower strand an oligonucleotide in which the phosphate group is replaced by a nitrogen group were also found to improve ligation specificity, although the degree of improvement was found to be less than with the adaptors described above.

In addition, ligation conditions which conferred high reaction efficiency were used (as described in materials and methods).

Again taking advantage of the solid support, the cDNA was then purified to remove excess non-ligated adaptor. PCR was performed on the 256 fractions using one universal primer complementary to the constant part of the adapter sequence and one complementary to the poly-A tail.

The 3′ primers were oligo dT and therefore complementary to the polyadenylation sequence of the original mRNA. Each primer was designed with a base extending into unknown sequence, guanine, adenosine or cytosine. (A second or still further base may be included, being any of guanine, adenosine, thymine or cytosine.) Each well received a mixture of the three possible 3′ primers. This ensured that the 3′ primer would always direct the polymerase to the beginning of the poly-A tail, giving a defined and reproducible fragment length.

The advantage of this second protocol is that the splitting into multiple frames occurs at the ligation step, not the PCR, allowing the use of high-stringency universal primers in the PCR. This leads to improved specificity and reproducibility. Another advantage is that a set of 256 adapters compatible with any 4-base overhang can be reused in multiple experiments with Type IIS enzymes which recognize different sequences but still give four base overhangs. Thus for each length of overhang, a single set of adapters will suffice.

The resulting PCR products were purified and loaded onto an ABI prism capillary sequencer. The PCR fragments representing the expressed genes were thus separated according to size and the fluorescence of each fragment quantified using the detector and software supplied with the ABI Prism.

Four separate frames may be run in each reaction vessel using different fluorophores because the ABI Prism has four detection channels. Four different universal forward primers (5′ end) have been designed with no cross-hybridization between them. The use of these primers allowed the 256 reactions to be reduced to 64. In an alternative embodiment, three primers and three adaptors are employed, allowing for one channel in the ABI Prism to be used for a size reference. The total number of reactions is then 86.

It is also desirable to increase the annealing temperature of the oligo-dT primer. This was enabled by adding a tail with an arbitrary sequence (not cross-hybridizing with any of the forward primers) and mixing the long primer containing oligo-dT with a short primer identical with the arbitrary sequence and having a high melting point. The first few cycles were then be performed at low temperature, at which only the oligo-dT primers anneal, after which all fragments had the tail added. This then allowed for subsequent cycles to be performed at higher temperature (at which only the short primer anneals) relying on the longer tail being present. This approach increases specificity of PCR and reduces background.

The combination of primers used leads to a theoretical mean of ˜80 PCR products in each fluorescent channel and sample (based on 20% genes expressed in a given sample and a total of 100 000 transcripts). Analysis of statistical size distribution of 3′ fragments including the polyadenylation generated from known genes following FokI restriction digestion, provides that an estimated 67% can be uniquely identified based on frame and length of fragment alone. Using an additional parallel experiment using the same protocol but replacing the FokI enzyme with another 5 base cutting class IIS restriction enzyme increases the theoretical limit to ˜89%; a third experiment yields ˜99% of all transcripts in the genome.

These numbers are under-estimates since in practice a gene that runs as a doublet in two experiments can still be identified as unique if at least one of its doublet partners is not expressed (a 96% chance) using combinatorial algorithms in accordance with the present invention. This and similar effects have been disregarded in the above calculations.

Combining the information unique to each fragment in this analysis, i.e. 9 nucleotides (including the FokI recognition sequence and cleavage site) and the size from polyadenylation to the FokI restriction site obtained from the capillary sequencer, the identity of each gene fragment (each corresponding uniquely to an mRNA in the sample) can thus be established by comparison with a database of RNA's of known polyA sites and/or virtual genes, as discussed.

Fragment Identification

Combinatorial algorithms of the invention, based on multiple independent patterns for a sample, offer a number of advantages for gene identification.

Firstly, the more experiments are performed the likelier it is that a given gene runs as a singlet fragment in at least one of them and can thus be unambiguously identified. Even if a given gene runs as a doublet in all experiments, it can still be identified if one of its doublet partners in one of the experiments should run as a singlet in another experiment and is absent there.

For example, if there is a fragment in experiment I at 162 bp corresponding to genes A and B, and one in experiment II at 367 bp corresponding to A and C, then one can look up C in experiment I (if it should run as a singlet there, say at 214 bp, and it is absent, i.e. there is no peak at 214 bp, then the peak at 162 bp in I can be identified as A) and B in experiment II. This simple procedure greatly increases the number of genes which can be unambiguously identified even when only two experiments have been performed.

Computer simulations using estimated error rates from an ABI Prism capillary electrophoresis machine indicate that 85-99% of all genes can be correctly identified even in the presence of normal fragment length errors.

Secondly, both of these combinatorial algorithms can be used to overcome uncertainties about fragment sizes or gene 3′-end lengths. This is because as long as the number of fragment peaks obtained from the sample plus the number of genes which can be eliminated as definitely not expressed is greater than the total number of candidate genes (i.e., the number of genes in the organism), the algorithms will be successful in assigning a gene to each fragment. In terms of the mathematical form of the algorithm, the system can be solved if the number of equations is greater than the number of candidate genes.

Thus, the number of candidate genes can be increased, up to a point, without losing the ability to successfully choose the correct candidate for each fragment. In cases where the length of the fragment is unknown, matches to fragments having each of the possible fragment lengths can be added to the list of genes which may be present. Similarly, when the position of the 3′ end in the database is unknown, all genes which could have a 3′ end in the position indicated by the fragment can be added to the list of genes which may be present. The false positives are subsequently eliminated automatically by the algorithm, provided the above condition is fulfilled.

The power of the system to eliminate false positives can be increased by performing greater numbers of independent profiles, as this will increase both the number of fragments and the number of genes which can be eliminated as definitely not present.

The optimum number of subdivisions can be determined.

The purpose of subdividing the reaction is to reduce the number of fragment peaks which correspond to multiple genes.

Two factors determine the number of doublets: the number of sub-reactions and the size distribution of fragments.

The optimal size distribution depends on the detection method. Capillary electrophoresis has single-basepair resolution up to 500 bp and about 0.15% resolution after that. Thus a distribution extending too far would not be useful. But a narrow distribution may present difficulties as well, because then genes will begin to run as true doublets (with the exact same length) which cannot be resolved no matter what the resolution.

The probability of finding a fragment of length n if you cut with an enzyme which cuts with a probability 1/512 is

P₁(n)=(511/512)ⁿ(1/512)

If the reaction is divided in 192 sub-reactions, the probability of finding a fragment of length n in a given subreaction is

P₂(n)=(511/512)ⁿ(1/512)(1/192)

The probability of this fragment corresponding to a single gene from M possible genes is

P_{unique}(n)=P₂(n)(1−P₂(n))^{(M-1)}

In other words, this is the probability that one gene gives a fragment of that length and all others do not.

The total number of genes which can be uniquely identified in a single experiment can be obtained by summing over all detectable lengths.

Taking instrument imprecision into account, P_{unique} becomes

P_{unique}(n)=P₂(n)((1−P₂(n))^{(M-1)})^{(1+2En)}

where E is the magnitude of the imprecision. This states that a unique gene can be identified if no other gene has the same length ± a factor E.

For example, if there are 50 000 genes in the human, our instrument has an error of 0.2% and can detect fragments up to 1000 bp, and we cut with an enzyme which cuts 1/512 of all sequences, subdividing in 192 subreactions, then we can identify 56% of all genes uniquely in a single experiment, 80% in two and 96% in three.

In Mathematica, the number of uniquely identifiable genes can be calculated as follows:

Prob[n_]:=(511/512)\hat{ }*1/512*1/192

Sum[50000*Prob[n]((1−Prob[n])\hat{ }50000)\hat{ }1+0.002n), {n,1,1000}]*192

By varying the parameters one can quickly see the effects on identification probabilities.

As noted above, if more experiments are performed, more powerful combinatorial identification methods can be used, but they all benefit from an increased number of singleton genes.

Materials and Methods

Section 1—Employing Type II Restriction Enzyme

Isolating mRNA from Total RNA

Isolate mRNA from 20 ug total RNA-according to Oligotex protocol until pure mRNA is bound to the beads and washed clean. Spin down and resuspend in 20 ul distilled water. The suspension should contain 0.5 mg Oligotex.

Split the reaction in 2×10 ul. Heat denature at 70° C. for 10 min, then chill quickly on ice. Synthesize first strand cDNA using each of the protocols below:

First Strand cDNA Synthesis Using AMV

Add first-strand buffer: 5 ul 5×AMV buffer, 2.5 ul 10 mM dNTP, 2.5 ul 40 mM NaPyrophosphate, 0.5 ul RNase inhibitor, 2 ul AMV RT, 2.5 ul 5 mg/ml BSA.

Incubate at 42° C. for 60 min. Total volume: 25 ul. [Note: it may be better to run in 100 ul, to get a more dilute oligotex suspension]

Second Strand cDNA Synthesis Using AMV

Add 12.5 ul 10×AMV second-strand buffer (500 mM Tris pH 7.2, 900 mM KCl, 30 mM MgCl₂, 30 mM DTT, 5 mg/ml BSA), 29 U E Coli DNA Polymerase I, 1 U RNase H to a final volume of 125 ul with dH₂O.

Incubate at 14° C. for 2 hours.

Restriction Enzyme Cleavage and Dephosphorylation

Spin down Oligotex/cDNA complexes and resuspend in 1.8 ul 1×FokI buffer, 16.2 ul H2O, 2 ul FokI, 1 u Calf Intestinal Phosphatase (included to dephosphorylate cohesive ends to prevent self-ligation in the next step).

Incubate at 37° C. for 1 hour.

Spin down and remove supernatant for quality-control.

Phosphatase Deactivation

Add 70 ul TE. Heat to 70° C. for 10 minutes. Cool down to room temperature and leave for 10 minutes.

Ligation

Resuspend in 2 ul 10× ligation buffer, 100× adaptor, 2 ul ligase, H₂O to 20 ul.

Incubate at RT for 2 hours.

Spin down and wash with 10 mM Tris (pH 7.6).

Primer and Adaptor Design

The adaptor is as follows (shown 5′ to 3′). It consists of a long and a short strand which are complementary. The long strand has four extra bases complementary to the GCGC cohesive end generated by the HaeII enzyme cleavage.

| | |
|---|---|
| 5′-GTCCTCGATGTGCGC-3′ | (SEQ ID NO. 1) |
| 5′-ACATCGAGGAC-3′ | (SEQ ID NO. 2) |

The 5′ primers are 5′-GTCCTCGATGTGCGCWN-3′ (SEQ ID NO. 3), where W is A or T and N is A, C, G or T. There are 8 different 5′ primers, labelled with a fluorochrome corresponding to the last base.

The 3′ primers are T₂₀VNN, where V is A, G or C and N is A, G, C or T. That is, 25 thymines followed by three bases as shown. There are 48 different 3′ primers.

All combinations of 3′ and 5′ primers are used, or 384 in total. The 5′ primers are pooled with respect to the last base (i.e. all four fluorochromes are run in the same reaction), giving a total of 96 reactions.

The primer combinations are predispensed into 96-well PCR plates.

PCR Amplification

Resuspend in 768 ul PCR buffer (buffer, enzyme, DNTP), add 8 ul to each well of a premade primer-plate containing 2 ul primer-mix (four 5′ primers and one 3′ primer) per well.

Using hot-start touchdown PCR, amplify each fraction as follows:

Hot start

Heat to 70° C.

Add Taq polymerase

10 cycles

94° C. 30 s

60° C. 30 s, reduced by 0.5° C. each cycle

72° C. 1 min

25 cycles

94° C. 30 s

55° C. 30 s

72° C. 1 min

Finally

72° C. 5 min

Cool down to 4° C.

The touchdown ramp annealing temperature may have to be adjusted up or down. The reaction should only proceed until the plateau phase has been reached; the 25 cycles may have to be adjusted.

Quantification by Capillary Electrophoresis

Load the 96-well plate on an ABI Prism 3700 setup for fragment analysis with a long capillary and long run time. The output is a table of fragment length (in base pairs) and peak height/area for each peak detected.

Proceed to identification, e.g. as described above with reference to a database.

Section 2—Employing Type IIS Restriction Enzyme

Preparation of Streptavidin Dynabeads (Attaching the Oligos to the Beads)

Wash 200 μl Dynabeads twice in 200 μl B&W buffer (Dynabeads) and then resuspend the beads in 400 μl B&W buffer.

Suspend 1250 pmol biotine T25 primer in 400 μl H₂O and mix with the beads. Incubate at RT for 15 min. Spin briefly, then remove 600 μl of the supernatent. Dispense the beads and place on a magnet for at least 30 seconds.

Wash beads twice with 200 μl B&W, and then resuspend in 200 μl B&W buffer.

Binding the mRNA to the Beads from Total RNA

Transfer 200 μl of resuspended beads into a 1.5 ml Eppendorf tube. Place on a magnet at least for 30 sec. Remove the supernatant and resuspend in 100 μl of binding buffer(20 mM Tris-HCl, pH 7,5; 1,0 M LiCl; 2 mM EDTA). Repeat washing, and resuspend the beads in 100μl of binding buffer.

Adjust ˜75 μg of total RNA or 2.5 μg of mRNA to 100 μl with Rnase free water or 10 mM Tris-HCl. Heat to 65° C. for 2 min.

Mix the beads thoroughly with the preheated RNA solution. Anneal by rotating or otherwise mixing for 3-5 min at room temperature (rt). Place on a magnet for at least 30 sec. Wash twice with 200 μl of washing buffer B (10 mM Tris-HCL pH7.5;0.15 MliCl; 1 mM EDTA).

First Strand Synthesis

Wash the beads at least twice with 200 μl 1 ×AMV buffer (Promega) using the magnet as described previously. Mix together 5 μl 5×AMV buffer; 2.5 μl 10 mM DNTP; 2.5 μl 40 mM Na pyrophosphate; 0.5 μl RNase inhibitor; 2 μl AMV RT (Promega); 1.25 μl 10 mg/ml BSA; 11.25 μl H₂O (Rnase free) (Total volume 25 μl). Resuspend the beads in this mixture.

Incubate at 42° C. for 1 h, with mixing.

Second Strand Synthesis

Add 100 μl of second strand mixture (6.25 μl 1M Tris pH 7.5; 11.25 μl 1M KCl; 15 μl MgCl₂; 3.75 μl DTT; 6.25 μl BSA; 1 μl Rnase H, 3 μl DNA pol I; 53.5 μl H₂O ) (total volume 100 μl) directly to the 1^{st} strand reaction.

Incubate at 14° C. for 2 h, with mixing.

Cleavage

Wash the beads on magnet 2× with TE (10 mM TRIS, 1 mM EDTA, pH 7.5) and 2× with 100-200 μl NEB buffer. Resuspend in 30 μl of NEB buffer.

Add 1 μl of the appropriate Type IIS enzyme and mix.

Incubate at 37° C. for 1-2 h, mixing frequently. Wash three times with TE in 1350 μl using the magnet as described above, and then twice with 1350 μl 2× ligation buffer.

Resuspend in 1606 μl 2× ligase buffer with ligase enzyme.

Adapter Ligation (in 256 Different Vessels)

Aliquot 6 μl of cut template per well in 256 wells containing 30 pmol adaptor in 4 μl for a total volume of 10 μl. Incubate 1 h at 37° C. with mixing. Wash in TE 80 μl 2× and dilute in 20 μl H₂O.

Adaptor and Primer Design

The adaptors in these embodiments are as follows (shown 5′ to 3′). Each pair is composed of a short and a long strand, which are complementary. The long strands have four nucleotides complementary to the cohesive ends generated by the FokI cleavage (a total of 4×4×4×4=256 possible adapters).

Labelled versions of the upper, shorter strands also serve as forward PCR primers.

| | |
|---|---|
| 5′-CCAAACCCGCTTATTCTCCGCAGTA-3′ | (SEQ ID NO. 4) |
| 5′-NNNNTACTGCGGAGAATAAGCGGGTTTGG-3′ | (SEQ ID NO. 5) |
| 5′-GTGCTCTGGTGCTACGCATTTACCG-3′ | (SEQ ID NO. 6) |
| 5′-NNNNCGGTAAATGCGTAGCACCAGAGCAC-3′ | (SEQ ID NO. 7) |
| 5′-CCGTGGCAATTAGTCGTCTAACGCT-3′ | (SEQ ID NO. 8) |
| 5′-NNNNAGCGTTAGACGACTAATTGCCACGG-3′ | (SEQ ID NO. 9) |

Each of the adaptors is be blocked on one strand. This may be achieved by blocking the upper strand at the 3′ end using a deoxy (dd) oligonucleotide, as shown below.

(SEQ ID NO. 4)

5′ (OH)-CCAAACCCGCTTATTCTCCGCAGTddA-3′

(SEQ ID NO. 5)

5′ (P)-NNNNTACTGCGGAGAATAAGCGGGTTTGG-(OH)3′

(SEQ ID NO. 6)

5′ (OH)-GTGCTCTGGTGCTACGCATTTACCddG-3′

(SEQ ID NO. 7)

5′ (P)-NNNNCGGTAAATGCGTAGCACCAGAGCAC-(OH)3′

(SEQ ID NO. 8)

5′ (OH)-CCGTGGCAATTAGTCGTCTAACGCddT-3′

(SEQ ID NO. 9)

5′ (P)-NNNNAGCGTTAGACGACTAATTGCCACGG-(OH)3′

Alternatively, blocking may be achieved by replacing the phosphate group at the 5′ end of the lower strand with a nitrogen, hydroxyl, or other blocking moiety.

The reverse primers are as follows

(SEQ ID NO. 10)

5′-CTGGGTAGGTCCGATTTAGGCTTTTTTTTTTTTTTTTTTTTTV-3′

(SEQ ID NO. 11)

5′-CTGGGTAGGTCCGATTTAGGC-3′

where V=A, C or G, for a total of three long reverse primers.

Universal PCR

Add 18 ul PCR buffer (buffer, enzyme, dNTP, three universal adapter primers, anchored oligo-T primers).

Amplify each fraction as follows:

Hot start

Heat

Add Taq at 70° C. (or use heat-activated Taq)

2 cycles

94° C. 30 s 50° C. 30 s 72° C. 1 min

25 cycles

94° C. 30 s 61° C. 30 s 72° C. 1 min

Finally

72° C. 5 min Cool down to 40° C.

Quantification by Capillary Electrophoresis

Load the 96-well plate on an ABI Prism 3700 setup for fragment analysis with a long capillary and long run time. The output will be a table of fragment length (in base pairs) and peak height/area for each peak detected.

References

Alizadeh et al. (2000) Nature 403, 503-511.

Alwine et al. (1977) Proc. Natl. Acad. Sci. USA 74, 5350-5354.

Beaudoing et al. (2000) Genome Res 10, 1001-10

Berk and Sharp (1977) Cell 12, 721-732.

Bowtell (1999) [published erratum appears in Nat Genet 1999 February;21(2):241]. Nat Genet 21, 25-32.

Britton-Davidian et al. (2000) Nature 403, 158.

Brown and Botstein (1999) Nat Genet 21, 33-7.

Cahill et al. (1999) Trends Cell Biol 9, M57-60.

Cho et al. (1998) Mol Cell 2, 65-73.

Collins et al. (1997) Science 278, 1580-1.

Der et al. (1998) Proc Natl Acad Sci USA 95, 15623-8.

Duggan et al. (1999) Nat Genet 21, 10-4.

Goldmann et al. (1999) J Gen Virol 80, 2275-83

Golub et al. (1999) Science 286, 531-7.

Iyer et al. (1999) Science 283, 83-7.

Kan et al. (2001) Genome Res 11, 889-900

Lander (1999) Nat Genet 21, 3-4.

Lengauer et al. (1998) Nature 396, 643-9.

Liang and Pardee (1992) Science 257, 967-71.

Lipshutz et al., (1999). High density synthetic oligonucleotide arrays. Nat Genet 21, 20-4.

McCormick (1999) Trends Cell Biol 9, M53-6.

Okubo et al. (1992) Nat Genet 2, 173-9.

Paabo (1999) Trends Cell Biol 9, M13-6.

Pauws et al. (2001) Nucl Acids Res 29, 1690-4

Perou et al. (1999) Proc Natl Acad Sci USA 96, 9212-7.

Schena et al. (1995) Science 270, 467-70.

Schena et al. (1996) Proc Natl Acad Sci USA 93, 10614-9.

Southern et al. (1999) Nat Genet 21, 5-9.

Stoler et al. (1999) Proc Natl Acad Sci USA 96, 15121-6.

Szallasi (1998) Nat Biotechnol 16, 1292-3.

Tabaska and Zhang (1999) Gene 231, 77-86

Thomson and Esposito (1999) Trends Cell Biol 9, M17-20.

Touriol et al. (1999) J Biol Chem 274, 21402-8

Velculescu et al. (1995) Science 270, 484-7.

1. A method for determining the presence of and/or identifying a polyadenylation site or alternative polyadenylation sites within a sequence of a transcribed gene or sequences of transcribed gene variants present or potentially present in a sample, the method comprising:
(a) generating a dataset comprising a set of signals obtained for individual gene fragments within a population of gene fragments produced from transcribed genes in the sample, wherein the signal for an individual gene fragment comprises a combination of length and partial sequence information and a magnitude component for that gene fragment, wherein the dataset contains a magnitude component of zero for combinations of length and partial sequence information determined not to be present in the population and the magnitude component of the signal for gene fragments for which the combination of length and partial sequence information is determined to be present is either qualitative to indicate presence in the population of a gene fragment with that combination or quantitative to provide an indication of the amount of individual gene fragments present in the population; and
(b) assigning to gene fragments one or more gene candidates within a database by comparing signals within the dataset with the database, the database comprising data representing mRNA's with known polyA sites and/or “virtual genes”, wherein virtual genes are defined as each representing a possible polyadenylation site within an actual gene,
(c) eliminating from results gene candidates which are each assigned to at least one signal of magnitude zero,
(d) thereby obtaining results defining a set of one or more genes or gene variants each being a mRNA with a known polyadenylation site and/or virtual gene assigned to a signal with non-zero magnitude in the dataset, which results provide indication of actual presence of said set of one or more genes or gene variants in said sample.

2. A method according to claim 1 wherein the virtual genes in the database are provided by scoring possible polyadenylation sites within an actual gene for likelihood of actual occurrence and including in the database virtual genes that exceed a defined threshold of likelihood of actual occurrence.

3. A method according to claim 1 wherein the virtual genes in the database collectively represent all possible polyadenylation sites within one or more actual genes.

4. A method according to any one of claims 1 to 3 wherein the population of gene fragments is provided by cutting cDNA copies of mRNA in a sample and purifying cut gene fragments that each comprise a terminal polyA sequence.

5. A method according to claim 4 wherein the population of gene fragments is provided by digesting with a restriction enzyme cDNA copies of mRNA in a sample and purifying digested gene fragments that each comprise a terminal polyA sequence.

6. A method according to claim 5 comprising
providing a first population of gene fragments by digesting with a first restriction enzyme cDNA copies of mRNA in a sample and purifying digested gene fragments that each comprise a terminal polyA sequence; and
providing a second population of gene fragments by digesting with a second restriction enzyme cDNA copies of mRNA in the sample and purifying digested gene fragments that each comprise a terminal polyA sequence; and optionally
providing a third population or further populations of gene fragments by digesting with a third restriction enzyme, or further restriction enzymes, cDNA copies of mRNA in the sample and purifying digested gene fragments that each comprise a terminal polyA sequence.

7. A method according to claim 6 comprising
determining the identity of one or more mRNA's with known polyA sites and/or virtual genes with a non-zero magnitude signal within signals for each of the first population and the second population, and optionally the third population or the further populations, within the dataset, whereby a mRNA with known polyA site and/or virtual gene that has a non-zero magnitude signal within the signals for both the first and second populations or all the populations is identified as corresponding to a polyadenylation site in a transcribed gene or transcribed gene variants present in the sample.

8. A method according to claim 6 or claim 7 wherein a first, second and third restriction enzyme are employed, providing first, second and third populations of gene fragments.

9. A method according to any one of claims 1 to 8 wherein the signal for a gene fragment comprises quantitative information on amount of the gene fragment present.

10. A method according to any one of claims 5 to 9 comprising:
synthesizing a cDNA strand complementary to each mRNA in the sample using the mRNA as template, thereby providing a population of first cDNA strands;
removing the mRNA;
synthesizing a second cDNA strand complementary to each first strand, thereby providing a population of double-stranded cDNA molecules;
digesting the double-stranded cDNA molecules with a Type II or Type IIS restriction enzyme to provide a population of digested double-stranded cDNA molecules, each digested double-stranded cDNA molecule having a cohesive end provided by the restriction enzyme digestion;
ligating a population of adaptor oligonucleotides to the cohesive end of each of the digested double-stranded cDNA molecules, the adaptor oligonucleotides each comprising an end sequence complementary to a cohesive end and a primer annealing sequence, thereby providing double-stranded template cDNA molecules each comprising a first strand and a second strand wherein the first strand of the double-stranded template cDNA molecules each comprise a 3′ terminal adaptor-oligonucleotide and the second strand of the double-stranded template cDNA molecules each comprise a 3′ terminal polyA sequence;
purifying said double-stranded template cDNA molecules;
performing polymerase chain reaction amplification on the double-stranded template cDNA molecules having a sequence complementary to a 3′ end of an mRNA using a population of first primers and a population of second primers,
wherein the first primers each comprise a sequence which anneals to a primer annealing sequence of an adaptor oligonucleotide; and
where the restriction enzyme is a Type II enzyme the first primers each comprise at least one 3′ terminal variable nucleotide and optionally more than one 3′ terminal variable nucleotides wherein the variable nucleotide is, or at a corresponding position within the variable nucleotides each first primer has, a nucleotide selected from A, T, C and G, whereby the population of first primers primes synthesis in the polymerase chain reaction of first strand product DNA molecules each of which is complementary to the first strand of a template cDNA molecule that comprises adjacent to the primer annealing sequence within the first strand of the template cDNA molecule a nucleotide or sequence of nucleotides complementary to the variable nucleotide or nucleotides of a first primer within the population of first primers; or
where the restriction enzyme is a Type IIS enzyme the first primers prime synthesis in the polymerase chain reaction of first strand product DNA molecules each of which is complementary to the first strand of a template cDNA molecule that comprises within the first strand of the template cDNA molecule a sequence of nucleotides complementary to an end sequence of an adaptor oligonucleotide in the population of adaptor oligonucleotides;
the second primers comprise an oligoT sequence and a 3′ variable portion conforming to the following formula: (G/C/A)(X)ₙ wherein X is any nucleotide, n is zero, at least one or more than one; whereby the population of second primers primes synthesis in the polymerase chain reaction of second strand product DNA molecules each of which is complementary to the second strand of a template cDNA molecule that comprises adjacent to polyA within the second strand of the template cDNA molecule a nucleotide or nucleotides complementary to the variable portion of a second primer within the population of second primers;
whereby the polymerase chain reaction amplification provides a population of double-stranded product DNA molecules (said gene fragments) each of which comprises a first strand product DNA molecule and a second strand product DNA molecule;
separating double-stranded product DNA molecules on the basis of length; and
detecting said double-stranded product DNA molecules;
whereby a signal for each double-stranded product DNA molecule is provided by combination of length of said double-stranded product DNA molecules and (i) first primer variable nucleotide or nucleotides, where a Type II restriction enzyme is employed, or (ii) adaptor oligonucleotide end sequence, where a Type IIS restriction enzyme is employed;
wherein signals are provided for first and second populations and optionally a third population or further populations of double-stranded product DNA molecules (said gene fragments) obtained by means of first and second different restriction enzymes and optionally a third different restriction enzyme or further different restriction enzymes.

11. A method according to any one of the preceding claims wherein signals in the dataset are compared with a database of signals determined or predicted for mRNA's with known polyA sites and/or said virtual genes, by:
(i) listing all mRNA's with known polyA sites and/or virtual genes in the database which may correspond to a gene fragment in each of said first and second and optionally third or further populations, forming a list of mRNA's with known polyA sites and/or virtual genes possibly present for each population, and
(ii) listing mRNA's with known polyA sites and/or virtual genes which definitely do not correspond to a gene fragment, forming a list of mRNA's with known polyA sites and/or virtual genes definitely not present for each population, then
(iii) removing the mRNA's with known polyA sites and/or virtual genes definitely not present from the list of mRNA's with known polyA sites and/or virtual genes possibly present for each population, and
(iv) generating a list of mRNA's with known polyA sites and/or virtual genes possibly present and mRNA molecules definitely not present by combining each list generated for each population in (iii);
thereby identifying one or more mRNA's with known polyA sites and/or virtual genes as corresponding to mRNA actually present in the sample.

12. A method according to claim 11 which comprises:
(i)listing all mRNA's of known polyA site and/or virtual gene in the database which may correspond to a gene fragment in each of the first and second and optionally third or further populations, and forming a set of equations of the form Fi=m₁+m₂+m₃, wherein Fi is the intensity of the signal from the fragment, the numerals are the identity of the mRNA's of known polyA sites and/or virtual genes in the database and wherein each mRNA with known polyA site or virtual gene which may correspond to a gene fragment appears as a term on the right-hand side;
(ii) for each experiment listing mRNA's of known polyA site and/or virtual genes which definitely do not correspond to a gene fragment in each population, and writing for each mRNA of known polyA site and/or virtual gene which definitely does not correspond to a gene fragment in each population an equation of the form 0=m₄, wherein the numeral is the identity of the mRNA of known polyA site and/or virtual gene in the database;
(iii) combining the sets of equations to form a system of simultaneous equations wherein the number of equations is greater than the number of transcribed genes or transcribed gene variants present or potentially present in the sample;
(iv) determining an amount of the expression level of each transcribed gene or transcribed gene variant by solving the system of simultaneous equations; and
(v) including the determined amounts of the expression levels within the signals provided for each gene fragment.

13. A method according to any one of claims 10 to 12, comprising purifying digested double-stranded cDNA molecules which comprise a strand comprising a 3′ terminal polyA sequence, prior to ligating the adaptor oligonucleotides.

14. A method according to claim 13, comprising:
i)immobilising mRNA molecules in the sample on a solid support by annealing a polyA tail of each mRNA molecule to polyT oligonucleotides attached to a support, prior to synthesizing said first cDNA strand, removing the mRNA, and synthesizing said second cDNA strand, thereby providing a population of double-stranded cDNA molecules attached to the support; and
ii) following digesting the double-stranded cDNA molecules to provide a population of digested double-stranded cDNA molecules attached to the support, purifying the digested double-stranded cDNA molecules attached to the support by washing away material not attached to the support, prior to ligating said population of adaptor oligonucleotides to the cohesive end of each of the digested double-stranded cDNA molecules; and
iii) following ligating a population of adaptor oligonucleotides to the cohesive end of each of the digested double-stranded cDNA molecules to provide said double-stranded cDNA template molecules, purifying the double-stranded template cDNA molecules by washing away material not attached to the support, prior to performing said polymerase chain reaction amplification on the double-stranded cDNA molecules.

15. A method according to any one claims 5 to 14 wherein the restriction enzyme cuts double-stranded DNA with a frequency of cutting of 1/256-1/4096 bp.

16. A method according to claim 15 wherein the frequency of cutting is 1/512 or 1/1024 bp.

17. A method according to any one claims 5 to 16 wherein the restriction enzyme is a Type II restriction enzyme.

18. A method according to claim 17 wherein the restriction enzyme digests double-stranded DNA to provide a cohesive end of 2-4 nucleotides.

19. A method according to claim 18 wherein the restriction enzyme is selected from the group consisting of HaeII, ApoI, XhoII and Hsp 921.

20. A method according to any one claims 17 to 19 wherein the first primers each have one variable nucleotide.

21. A method according to any one of claims 17 to 20 wherein the first primers each have two variable nucleotides, each of which may be A, T, C or G.

22. A method according to any one of claims 17 to 19 wherein the first primers each have three variable nucleotides, each of which may be A, T, C or G.

23. A method according to any one of claims 17 to 22 wherein each first primer is labelled with a label to indicate which of A, T, C and G is said variable nucleotide or is present at said corresponding position within the variable nucleotides of the first primer.

24. A method according to any one of claims 5 to 16 wherein the restriction enzyme is a Type IIS restriction enzyme.

25. A method according to claim 24 wherein the restriction enzyme digests double-stranded DNA to provide a cohesive end of 2-4 nucleotides.

26. A method according to claim 25 wherein the restriction enzyme is selected from the group consisting of FokI, BbvI, SfaNI and Alw261.

27. A method according to any one of claims 24 to 26 wherein adaptor oligonucleotides in the population of adaptor oligonucleotides are ligated to cohesive ends of digested double-stranded cDNA molecules in separate reaction vessels from different adaptor oligonucleotides with different end sequences.

28. A method according to claim 27 wherein each reaction vessel contains a single adaptor oligonucleotide end sequence.

29. A method according to claim 27 wherein each reaction vessel contains multiple adaptor oligonucleotide end sequences, each adaptor oligonucleotide sequence in a reaction vessel comprising a different end sequence and primer annealing sequence from the end sequence and primer annealing sequence of other adaptor oligonucleotide sequences in the same reaction vessel, corresponding multiple first primers being employed in the polymerase chain reaction amplification in each reaction vessel.

30. A method according to any one of claims 5 to 29 wherein n is 0.

31. A method according to any one of claims 5 to 29 wherein n is 1.

32. A method according to any one of claims 5 to 29 wherein n is 2.

33. A method according to any one claims 5 to 29 wherein first primers are labelled.

34. A method according to claim 33 wherein the labels are fluorescent dyes readable by a sequencing machine.

35. A method according to any one of claims 5 to 34 wherein double-stranded DNA molecules are separated on the basis of length by electrophoresis on a sequencing gel or capillary, and signals for gene fragments are generated as an electropherogram.


SEQUENCE LISTING (25 sequences)

SEQ ID NO: 1 - DNA, 15 bp, Artificial Sequence
  Description of Artificial Sequence Adaptor
gtcctcgatgtgcgc

SEQ ID NO: 2 - DNA, 11 bp, Artificial Sequence
  Description of Artificial Sequence Adaptor
acatcgaggac

SEQ ID NO: 3 - DNA, 17 bp, Artificial Sequence
  Description of Artificial Sequence Primer
gtcctcgatgtgcgcwn

SEQ ID NO: 4 - DNA, 25 bp, Artificial Sequence
  Description of Artificial Sequence Adaptor
ccaaacccgcttattctccgcagta

SEQ ID NO: 5 - DNA, 29 bp, Artificial Sequence
  Description of Artificial Sequence Adaptor
nnnntactgcggagaataagcgggtttgg

SEQ ID NO: 6 - DNA, 25 bp, Artificial Sequence
  Description of Artificial Sequence Adaptor
gtgctctggtgctacgcatttaccg

SEQ ID NO: 7 - DNA, 29 bp, Artificial Sequence
  Description of Artificial Sequence Adaptor
nnnncggtaaatgcgtagcaccagagcac

SEQ ID NO: 8 - DNA, 25 bp, Artificial Sequence
  Description of Artificial Sequence Adaptor
ccgtggcaattagtcgtctaacgct

SEQ ID NO: 9 - DNA, 29 bp, Artificial Sequence
  Description of Artificial Sequence Adaptor
nnnnagcgttagacgactaattgccacgg

SEQ ID NO: 10 - DNA, 43 bp, Artificial Sequence
  Description of Artificial Sequence Primer
ctgggtaggtccgatttaggctttttttttttttttttttttv

SEQ ID NO: 11 - DNA, 21 bp, Artificial Sequence
  Description of Artificial Sequence Primer
ctgggtaggtccgatttaggc

SEQ ID NO: 12 - DNA, 14 bp, Artificial Sequence
  Description of Artificial Sequence Digested double-stranded DNA
cgcgaacgcgtacg

SEQ ID NO: 13 - DNA, 10 bp, Artificial Sequence
  Description of Artificial Sequence Digested double-stranded DNA
cgtacgcgtt

SEQ ID NO: 14 - DNA, 25 bp, Artificial Sequence
  Description of Artificial Sequence Adaptor
acgcatttaccgcgcgacgcgtacg

SEQ ID NO: 15 - DNA, 25 bp, Artificial Sequence
  Description of Artificial Sequence Adaptor
cgtacgcgtcgcgcggtaaatgcgt

SEQ ID NO: 16 - DNA, 30 bp, Artificial Sequence
  Description of Artificial Sequence Double-stranded product DNA
catcagatacgtagcgaaaaaaaaaaaaaa

SEQ ID NO: 17 - DNA, 32 bp, Artificial Sequence
  Description of Artificial Sequence Double-stranded product DNA
ttttttttttttttttcgctacgtatctgatg

SEQ ID NO: 18 - DNA, 18 bp, Artificial Sequence
  Description of Artificial Sequence Double-stranded product DNA
ttttttttttttttttcg

SEQ ID NO: 19 - DNA, 19 bp, Artificial Sequence
  Description of Artificial Sequence Double-stranded product DNA
acgcatttaccgcgcgacg

SEQ ID NO: 20 - DNA, 18 bp, Artificial Sequence
  Description of Artificial Sequence Digested double-stranded DNA
cgctacgcgtacggtagg

SEQ ID NO: 21 - DNA, 14 bp, Artificial Sequence
  Description of Artificial Sequence Digested double-stranded DNA
cctaccgtacgcgt

SEQ ID NO: 22 - DNA, 25 bp, Artificial Sequence
  Description of Artificial Sequence Adaptor
acgcatttaccgcgctacgcgtacg

SEQ ID NO: 23 - DNA, 25 bp, Artificial Sequence
  Description of Artificial Sequence Adaptor
cgtacgcgtagcgcggtaaatgcgt

SEQ ID NO: 24 - DNA, 17 bp, Artificial Sequence
  Description of Artificial Sequence Double-stranded product DNA
ttttttttttttttttc

SEQ ID NO: 25 - DNA, 12 bp, Artificial Sequence
  Description of Artificial Sequence Double-stranded product DNA
acgcatttaccg